(19) United States
(12) Patent Application Publication
(10) Pub. No.: US 2025/0114162 A1
(43) Pub. Date: Apr. 10, 2025

(54) BLOCKS, SYSTEMS AND METHODS FOR DENTAL STABILISATION

(71) Applicant: DIGICUTO, Marseille (FR)

(72) Inventors: Stefan KOUBI, Marseille (FR); Karim TOURBAH, Marseille (FR); Galip GUREL, Marseille (FR); Maxence PINTA, Marseille (FR)

(21) Appl. No.: 18/838,084

(22) PCT Filed: Jun. 12, 2023
(86) PCT No.: PCT/EP2023/065691

§ 371 Date: Aug. 13, 2024

(30) Foreign Application Priority Data
Jul. 12, 2022 (FR) FR2207123

(51) Int. Cl. A61B 90/16

(52) U.S. Cl. CPC A61B 90/16

(57) ABSTRACT

One of the aims of the invention is to provide a dental stabilisation device which prevents a patient, during a dental procedure, from easily releasing their teeth from said device. To this end, the inventor proposes a dental stabilisation block which comprises internal channels that open onto the faces of the dental stabilisation block which come into contact with the patient's teeth. These internal channels are connected to an inlet via which a bite registration material can be injected, said material being intended to harden and be moulded to the patient's teeth. In this way, when the bite registration material has hardened and has moulded around the teeth, it becomes more difficult for the patient to release their teeth from the dental stabilisation block.

### TECHNICAL FIELD

The invention relates to the field of dental procedures. Particularly, it relates to dental stabilization blocks, dental stabilization systems and dental stabilization methods for keeping the patient's mouth open while preventing the teeth from meeting.

Similar devices are known from document US2018/125349A1.

### PRIOR ART

In the dental field, dental stabilization devices (bite blocks) are known, which are also referred to by the following terms: “jaw support”, “bitten block”, “bite piece”, or “bite block”.

However, known dental stabilization devices allow the patient to easily release their mouth from the device.

However, within the framework of some dental procedures, it is essential that the patient does not release their teeth from the dental stabilization device.

Thus, there is a need for a solution that avoids the situation described above.

### SUMMARY OF THE INVENTION

The invention aims to address, at least partially, this need.

A first aspect of the invention concerns a dental stabilization block which is intended to be bitten, only on one side of the mouth, between the upper and lower molars and/or premolars of a patient, called teeth, to keep the patient's mouth open during a dental procedure so as to prevent the teeth from meeting, thereby authorizing access to teeth and/or gums on the opposite side of the mouth and at the front of the mouth, without being substantially hampered by the dental stabilization block.

The dental stabilization block comprises:
a body, of essentially parallelepiped shape, which comprises, when the dental stabilization block is in place in the patient's mouth,
an upper wall, having an upper outer tightening surface which is designed to come into contact with the upper teeth, the upper outer tightening surface comprising at least a first outlet orifice,
a lower wall, having a lower outer tightening surface which is designed to come into contact with the lower teeth, the lower outer tightening surface comprising at least a second outlet orifice,
a first side wall which is oriented towards the inside of the mouth,
a second side wall which is oriented towards an inner face of the cheek,
a front end wall which is oriented towards the front of the mouth,
a rear end wall which is oriented towards the back of the mouth,
at least a first inner channel which opens out towards the outside of the body at the level of at least the first outlet orifice and/or at least the second outlet orifice, the first inner channel defining a flow path which is configured to be traversed by a bite registration material intended to harden and to be molded at least on the teeth,
at least a second inner channel which opens out towards the outside of the body at the level of an inlet orifice which is configured to receive at least one end portion of a device for injecting the bite registration material, the inlet orifice being disposed on an outer surface of the first side wall and/or the front end wall, and
a first inner junction region which is configured to establish fluid communication between the first inner channel and the second inner channel.

In a first embodiment, the dental stabilization block further comprises:
a plurality of first inner channels which is arranged so that, in response to the injection of the bite registration material, the bite registration material arrives substantially at the same time at the level of the outlet orifices, and
a plurality of second inner junction regions which is arranged so that each second inner junction region establishes fluid communication between at least two first inner channels.

In a second embodiment, the bite registration material is chosen from: waxes, zinc oxide-eugenol pastes, addition silicone elastomers, polyester elastomers and any combinations thereof.

In a third embodiment, the first side wall and the second side wall are designed to each extend towards the top and/or towards the bottom of the mouth beyond the upper outer tightening surface and/or the lower outer tightening surface to cover the upper and/or lower teeth so as to form an upper enclosure and/or a lower enclosure of U-shaped section in which the bite registration material and upper and/or lower teeth can be retained, the base of the U-shaped section of the upper enclosure or of the lower enclosure containing, respectively, the upper outer tightening surface or the lower outer tightening surface, the wings of the U-shaped section of the upper enclosure or of the lower enclosure containing, respectively, the first side wall and the second side wall.

In a fourth embodiment, the second side wall is provided with a cheek retractor part which is intended to retract the inner cheek face of the second side wall, the cheek retractor part protruding from the second side wall towards the inner cheek face and, when the dental stabilization block is in place in the patient's mouth, has an L shape with a rounded bend, whose one of the branches, called bearing branch, and the rounded bend bear on the inner cheek face and whose other branch, called cantilever branch, is cantilevered and bears, at least partly, on the rear end wall.

In one implementation of the fourth embodiment, the cheek retractor part comprises at least a third inner channel which is arranged, at least partly, inside the rounded bend, the third inner channel opening out towards the outside of the body of the bearing branch at the level of a suction orifice and at the level of a discharge orifice, the suction orifice being disposed on the cantilevered branch, the discharge orifice being disposed on the bearing branch.

In one example of the implementation of the fourth embodiment, the bearing branch comprises a suction cannula guide part which is open in the direction of the second side wall and which extends axially from the discharge orifice to the free end of the bearing branch.

In a fifth embodiment, the upper outer tightening surface and the lower outer tightening surface are provided with a plurality of fluid flow disturbance ribs.

In a sixth embodiment, when it depends on the fourth embodiment, the dental stabilization block further comprises an extension branch which extends obliquely or perpendicularly from the free end of the bearing branch towards the outside of the mouth, the extension branch being designed to form a direction change region which, when the extension branch deflects to come out of the patient's mouth, has an external surface that covers the labial commissure of the mouth,
in which,
at least one free end region of the extension branch comprises at least one support element which is adapted to support at least one position reference element which is designed to serve as a guide element for a navigation system for dental surgery, the position reference element being adapted, when the position reference element is moved in response to at least one movement of the patient's head, to trigger a registration of one or several dental images associated with the patient, based on the movement of the patient's head.

The invention also relates to a dental stabilization system comprising:
a dental stabilization block according to the first aspect of the invention, and
a position reference element which is configured to be coupled to at least one support element of the free end region of the extension branch, the position reference element being designed to serve as a guide element for a navigation system for dental surgery, the position reference element being adapted, when the position reference element is moved in response to at least one movement of the patient's head, to trigger a registration of one or several dental images associated with the patient, based on the movement of the patient's head.

In a first embodiment, the dental stabilization system comprises:
two dental stabilization blocks according to the first aspect of the invention, the dental stabilization blocks being configured to be positioned on opposite sides of the patient's mouth.

The invention finally relates to a method for dental stabilization during a dental procedure.

The method comprises:
a step of installing, in the patient's mouth, at least one dental stabilization block according to the first aspect of the invention, on upper and lower molars and/or premolars of a patient, called teeth,
a step of introducing an end portion of a device for injecting a bite registration material to the inlet orifice, the bite registration material being intended to harden and to be molded at least on the teeth, and
a step of injecting the bite registration material into the inlet orifice.

### BRIEF DESCRIPTION OF THE DRAWINGS

Other characteristics and advantages of the invention will be better understood upon reading the following description and with reference to the appended drawings, given by way of illustration and without limitation.

FIG. 1 represents a first perspective view of a dental stabilization block according to the invention.

FIG. 2 represents the dental stabilization block of [FIG. 1], when it is in place in a patient's mouth.

FIG. 3 represents a second perspective view of a dental stabilization block according to the invention.

FIG. 4 represents an enlarged view of [FIG. 1].

FIG. 5 represents a first sectional view of a dental stabilization block according to the invention.

FIG. 6 represents an enlarged view of [FIG. 3].

FIG. 7 represents a top view of [FIG. 3].

FIG. 8 represents a second sectional view of a dental stabilization block according to the invention.

FIG. 9 represents a top view of one embodiment of the invention.

FIG. 10 represents a method according to the invention.

### DESCRIPTION OF THE EMBODIMENTS

One of the objectives of this invention is to provide a dental stabilization device which prevents a patient, during a dental procedure, from easily releasing their mouth from the device.

For this, the inventor proposes a dental stabilization block which comprises inner channels which open out onto the faces of the dental stabilization block which come into contact with the patient's teeth. These inner channels are connected to an inlet through which a bite registration material which is intended to harden and to be molded onto the patient's teeth can be injected.

In this way, when the bite registration material has hardened and molded around the teeth, it becomes more difficult for the patient to release their teeth from the dental stabilizing block.

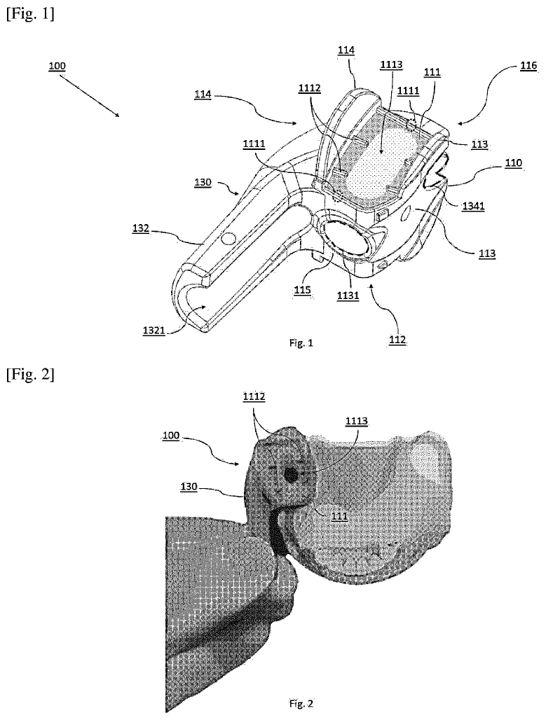

Thus, as illustrated in [FIG. 1], the invention relates to a dental stabilization block 100.

In practice, the dental stabilization block 100 is intended to be bitten, on one side of the mouth only, between the upper and lower molars and/or premolars of a patient, hereinafter called “teeth”.

In other words, when the dental stabilization block 100 is in place in the patient's mouth, the patient can bite the dental stabilization block 100, either with their upper and lower molars only, or with their upper and lower premolars only or with both their upper and lower molars and upper and lower premolars.

In one preferred embodiment of the invention, different sizes of the dental stabilization block 100 can be provided depending on the dental morphology of the patient, namely the arrangement of their teeth on the upper jaw and on the lower jaw.

For example, a model for children and a model for adults could be provided.

However, depending on the needs, other arrangements taking into account the dental morphology of the patients could be envisaged, without requiring substantial modifications to the invention.

FIG. 2 illustrates the dental stabilization block 100 when positioned on lower teeth located in the left side of the patient's mouth.

In practice, the dental stabilization block 100 makes it possible to keep the patient's mouth open during a dental procedure so as to prevent the teeth from meeting.

Thus, the dental stabilization block 100 authorizes access to teeth and gums located on the opposite side of the mouth and at the front of the mouth, without being significantly hampered by the dental stabilization block 100.

In other words, when the dental stabilization block 100 is in place in the patient's mouth, the practitioner who performs the dental procedure can access, inside the mouth, the teeth and gums which are in the front part of the mouth as well as the part of the mouth which is located opposite to the position of the dental stabilization block 100.

Thus, in a example, first if the dental stabilization block 100 is positioned on teeth located in the right side of the patient's mouth, then the practitioner can access the teeth and gums which are disposed at the front of the mouth as well as the teeth disposed to the left of the mouth.

Likewise, in a second example, if the dental stabilization block 100 is positioned on teeth located in the left side of the patient's mouth, then the practitioner can access the teeth and gums which are disposed at the front of the mouth as well as the teeth disposed to the right of the mouth.

Returning to the invention, the dental stabilization block 100 comprises a body 110 which is of essentially parallelepiped shape.

Thus, the body 110 has the shape of a parallelepiped, namely the shape of a six-face polyhedron whose six faces are parallel in pairs.

In practice, the shape of the body 110 is substantially curved to accommodate the approximate curve of the upper and lower teeth in a human jaw.

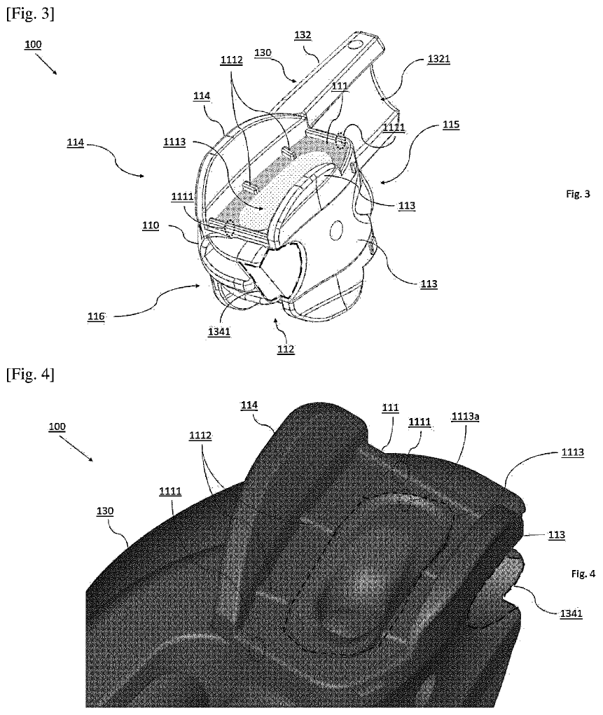

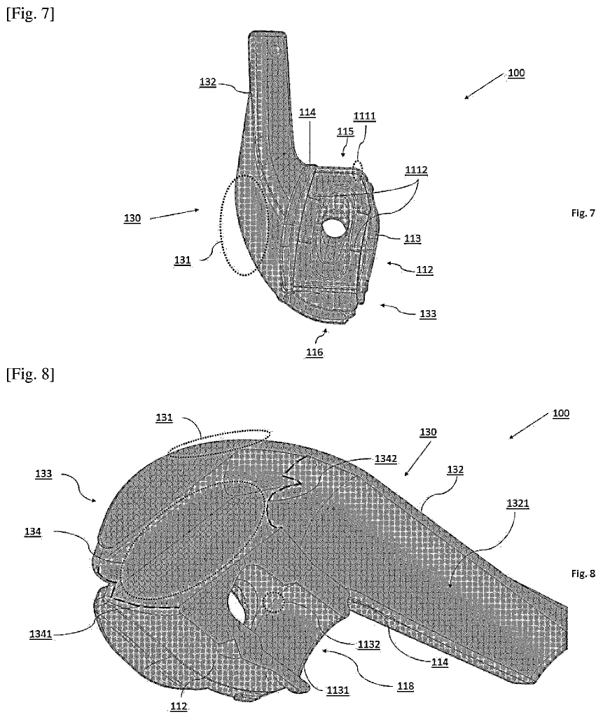

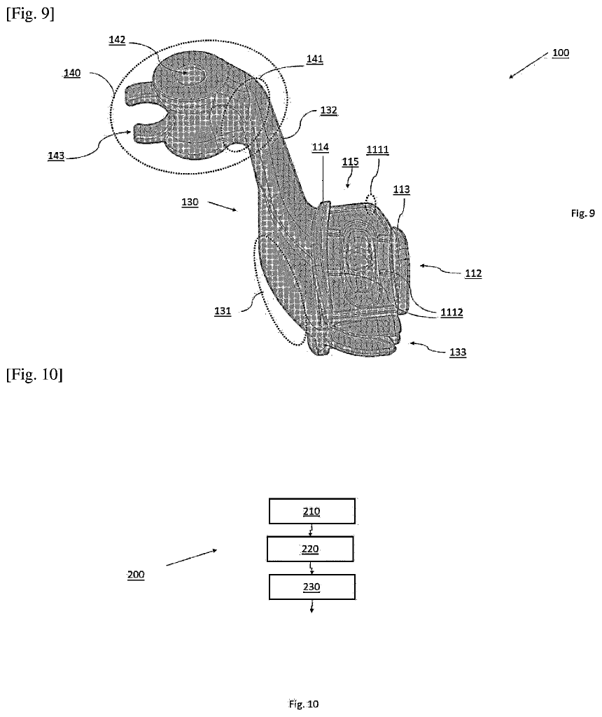

Particularly, as illustrated in [FIG. 1], [FIG. 3], [FIG. 7] and [FIG. 9], when the dental stabilization block 100 is in place in the patient's mouth, the body 110 comprises an upper wall 111, a lower wall 112, a first side wall 113, a second side wall 114, a front end wall 115 and a rear end wall 116.

In practice, the upper wall 111 has an upper outer tightening surface which is designed to come into contact with the upper teeth.

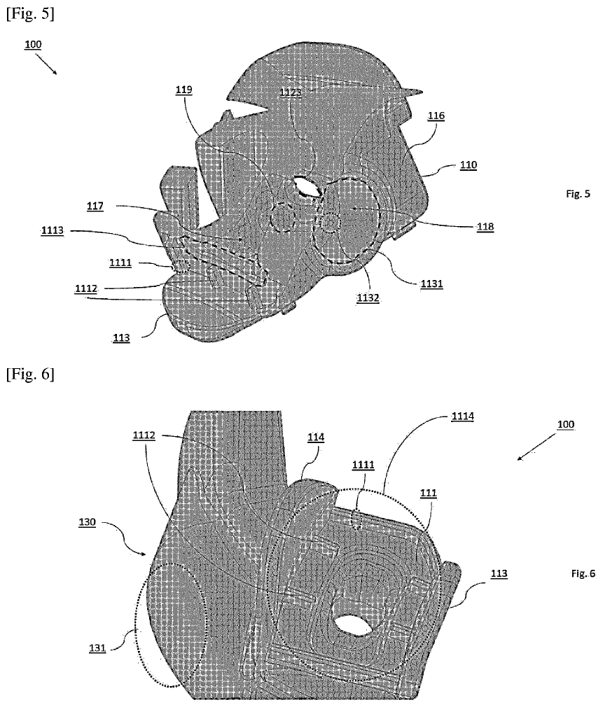

In one particular implementation of the upper outer tightening surface, as illustrated in [FIG. 1], [FIG. 3], [FIG. 4], [FIG. 5], [FIG. 6], [FIG. 7] and [FIG. 9], the upper outer tightening surface comprises an upper peripheral rim 1111.

Particularly, the upper peripheral rim 1111 is configured to retain a bite registration material as described below.

In one particular implementation of the upper outer tightening surface, as illustrated in [FIG. 1], [FIG. 3], [FIG. 4], [FIG. 5], [FIG. 6], [FIG. 7] and [FIG. 9], the latter is provided with a plurality of fluid flow disturbance ribs 1112.

In practice, each fluid flow disturbance rib 1112 is configured, when the dental stabilization block 100 is in place in the patient's mouth, to cause a disturbance of the flow of a fluid flowing over the upper outer tightening surface.

In a first example of this particular implementation, the fluid flow disturbance ribs 1112 extend towards the top from the upper outer tightening surface.

In a second example of this particular implementation, the fluid flow disturbance ribs 1112 extend through a fluid flow axis of the upper outer tightening surface.

In a third example of this particular implementation, the fluid flow disturbance ribs 1112 have an upper external turbulence surface which is chosen from: a stepped surface, a ridged surface, a dimpled surface or a rough surface.

Furthermore, as illustrated in [FIG. 1], [FIG. 3], [FIG. 4] and [FIG. 5], the upper outer tightening surface comprises at least a first outlet orifice 1113.

In one particular implementation of the first outlet orifice 1113, as illustrated in [FIG. 4], the first outlet orifice 1113 comprises a peripheral internal recess 1113a so as to form at least a first peripheral uncoupling surface which is arranged below the upper outer tightening surface.

Similarly to the upper wall 111, the lower wall 112 has a lower outer tightening surface which is designed to come into contact with the lower teeth.

In one particular implementation of the lower outer tightening surface, the latter is provided with a plurality of fluid flow disturbance ribs which are similar to the fluid flow disturbance ribs 1112 of the upper outer tightening surface.

In a first example of this particular implementation, the fluid flow disturbance ribs extend downwards from the lower outer tightening surface.

In a second example of this particular implementation, the fluid flow disturbance ribs extend through a fluid flow axis of the lower outer tightening surface.

In a third example of this particular implementation, the fluid flow disturbance ribs have a lower external turbulence surface which is chosen from: a stepped surface, a ridged surface, a dimpled surface or a rough surface.

Furthermore, the lower outer tightening surface comprises at least a second outlet orifice 1123.

As illustrated in [FIG. 5], similarly to the first outlet orifice 1113, in one particular implementation of the second outlet orifice 1123, the latter comprises a peripheral internal recess so as to form at least a second peripheral uncoupling surface which is arranged below the lower outer tightening surface.

As illustrated in [FIG. 2], in relation to [FIG. 1], [FIG. 3], [FIG. 4], [FIG. 5], [FIG. 6], [FIG. 7] and [FIG. 9], when the dental stabilization block 100 is in place in the patient's mouth:
the first side wall 113 is oriented towards the inside of the mouth,
the second side wall 114 is oriented towards an inner cheek face, particularly the inner cheek face located on the side of the mouth where the dental stabilization block 100 is installed. Thus, if the dental stabilization block 100 is installed in the right side of the patient's mouth, then the second side wall 114 is oriented towards the inner face of the patient's right cheek. Moreover, if the dental stabilization block 100 is installed in the left side of the patient's mouth, then the second side wall 114 is oriented towards the inner face of the patient's left cheek,
the front end wall 115 is oriented towards the front of the mouth, and
the rear end wall 116 is oriented towards the back of the mouth.

Furthermore, as illustrated in [FIG. 5], the body 110 comprises at least a first inner channel 117, at least a second inner channel 118 and a first inner junction region 119.

Particularly, the first inner channel 117, which is located inside the body 110, opens out towards the outside of the body 110 at the level of at least a first outlet orifice 1113 and/or at least a second outlet orifice 1123.

In other words, the first inner channel 117 can be opened out towards the outside, either by passing only through the first outlet orifice 1113, or by passing only through the second outlet orifice 1123 or by passing both through the first outlet orifice 1113 and the second outlet orifice 1123.

Moreover, the first inner channel 117 defines a flow path which is configured to be traversed by a bite registration material intended to harden and to be molded at least on the teeth.

In a first implementation of the bite registration material, the latter is chosen from waxes.

For example, it is possible to chose from mineral waxes (e.g. alkanes like paraffins), waxes of natural origin (e.g. esters like Carnauba waxes, beeswaxes), waxes of synthetic origin (e.g. polyethylene waxes, polyethylene glycol waxes, hydrogenated or halogenated hydrocarbon waxes) and any combinations thereof.

In a second implementation of the bite registration material, the latter is chosen from zinc oxide-eugenol pastes.

In a third implementation of the bite registration material, the latter is chosen from addition silicone elastomers.

In a fourth implementation of the bite registration material, the latter is chosen from polyester elastomers.

However, depending the needs, it could be envisaged to use other dental materials which are capable of deforming under occlusal stress, registering this deformation and restoring it without modifications, without requiring substantial modifications to the invention.

The second inner channel 118, which is located inside the body 110, opens out towards the outside of the body 110 at the level of an inlet orifice 1131.

The inlet orifice 1131 is configured to receive at least one end portion of a device for injecting the bite registration material.

In one example, the end portion according to the invention is a mixing tip of a bite registration injector of known type.

In one particular implementation of the inlet orifice 1131, as illustrated in [FIG. 5] and [FIG. 8], the inlet orifice 1131 comprises a shoulder 1132 against which the end of the device for injecting the bite registration material can come into abutment.

Furthermore, in the invention, the inlet orifice 1131 is disposed on an outer surface of the first side wall 113 and/or the front end wall 115.

In other words, the inlet orifice 1131 can be disposed either on an outer surface of the first side wall 113 only, or on an outer surface of the front end wall 115 only or on both an outer surface of the first side wall 113 and an outer surface of the front end wall 115.

Finally, the first inner junction region 119 is configured to establish fluid communication between the first inner channel 117 and the second inner channel 118.

Thus, when the bite registration material is injected into the inlet orifice 1131, the bite registration material can come out at the level of the outlet orifices 1113, 1123.

FIG. 5 illustrates the outlet orifices 1113, 1123.

In a first way of carrying out the invention, the body 110 comprises a plurality of first inner channels 117 and a plurality of second inner junction regions.

In practice, the plurality of first inner channels 117, which is located inside the body 110, is arranged so that, in response to the injection of the bite registration material, the bite registration material occurs substantially at the same time at the level of the outlet orifices 1113, 1123.

Furthermore, the plurality of second inner junction regions is arranged so that each second inner junction region establishes fluid communication between at least two first inner channels 117.

In other words, each second inner junction region can establish a fluid connection between two, three, four or more than four first inner channels 117.

In a second way of carrying out the invention, the first side wall 113 and the second side wall 114 are designed to each extend towards the top and/or towards the bottom of the mouth beyond the upper outer tightening surface and/or the lower outer tightening surface.

In other words, in the second way of carrying out the invention, the first side wall 113 and the second side wall 114 extend, either towards the top of the mouth only, or towards the bottom of the mouth only or both towards the top and the bottom of the mouth.

This arrangement is such that it allows, when the dental stabilization block 100 is in place in the patient's mouth, covering the upper and/or lower teeth so as to form an upper enclosure and/or a lower enclosure of U-shaped section in which the bite registration material and upper and/or lower teeth can be retained.

In other words, when the first side wall 113 and the second side wall 114 extend only towards the top of the mouth, they form the upper enclosure. Furthermore, when the first side wall 113 and the second side wall 114 extend only towards the bottom of the mouth, they form the lower enclosure. Finally, when the first side wall 113 and the second side wall 114 extend both towards the top and towards the bottom of the mouth, they form the upper enclosure and the lower enclosure.

Particularly, as illustrated in [FIG. 6], in the upper enclosure 1114 and in the lower enclosure, the base of the U-shaped section of the upper enclosure 1114 or of the lower enclosure contains, respectively, the upper outer tightening surface or the lower outer tightening surface.

Furthermore, the wings of the U-shaped section of the upper enclosure 1114 or of the lower enclosure contain, respectively, the first side wall 113 and the second side wall 114.

Thus, the upper enclosure 1114 has a U-shaped section, whose base contains the upper outer tightening surface and whose wings contain the first side wall 113 and the second side wall 114.

Moreover, similarly to the upper enclosure 1114, the lower enclosure has a U-shaped section, whose base contains the lower outer tightening surface and whose wings contain the first side wall 113 and the second side wall 114.

In one particular embodiment, as illustrated in [FIG. 1], [FIG. 3], [FIG. 4] and [FIG. 6], the second side wall 114 extends higher than the first side wall 113.

In a third way of carrying out the invention, the second side wall 114 is provided with a cheek retractor part 130 which is intended to retract the inner cheek face of the second side wall 114.

Particularly, as illustrated in [FIG. 1], [FIG. 2], [FIG. 3], [FIG. 6], [FIG. 7], [FIG. 8] and [FIG. 9], the cheek retractor portion 130 protrudes from the second side wall 114 towards the inner cheek face.

Furthermore, as illustrated in [FIG. 7] and [FIG. 9], when the dental stabilization block 100 is in place in the patient's mouth, the cheek retractor part 130 has an L shape with a rounded bend 131.

In practice, as illustrated in [FIG. 7] and [FIG. 9], one of the branches of the cheek retractor part 130, hereinafter called “bearing branch” 132 and the rounded bend 131 bear on the inner cheek face.

Moreover, the other branch of the cheek retractor part 130, hereinafter called “cantilever branch” 133 is cantilevered and bears, at least partly, on the rear end wall 116.

In a first embodiment of the third way of carrying out the invention, as illustrated in [FIG. 8], the cheek retractor portion 130 comprises at least a third inner channel 134 which is arranged, at least partly, inside the rounded bend 131.

In a first embodiment of the first embodiment of the third way of carrying out the invention, the inner channel 134 is arranged inside the rounded bend 131 and inside the bearing branch 132.

In a second embodiment of the first embodiment of the third way of carrying out the invention, the inner channel 134 is arranged inside the rounded bend 131 and inside the cantilever branch 133.

In a third embodiment of the first embodiment of the third way of carrying out the invention, the inner channel 134 is arranged inside the rounded bend 131, inside the bearing branch 132 and inside the cantilever branch 133.

In practice, the third inner channel 134 opens out towards the outside of the body of the bearing branch 132 at the level of a suction orifice 1341 and at the level of a discharge orifice 1342.

Particularly, as illustrated in [FIG. 8], the suction orifice 1341 is disposed on the cantilever branch 133 upstream of the rear end wall 116 relative to the patient's mouth.

Moreover, the discharge orifice 1342 is disposed on the bearing branch 132 downstream of the suction orifice 1341 relative to the patient's mouth.

In one particular implementation of the first embodiment of the third way of carrying out the invention, as illustrated in [FIG. 8], the bearing branch 132 comprises a suction cannula guide part 1321 which is open in the direction of the second side wall 114 and which extends axially from the discharge orifice 1342 to the free end of the bearing branch 132.

In a second embodiment of the third way of carrying out the invention, as illustrated in [FIG. 9], the dental stabilization block 100 further comprises an extension branch 140 which extends obliquely or perpendicularly from the free end of the bearing branch 132 towards the outside of the mouth.

In practice, the extension branch 140 is designed to form a direction change region 141 which, when the extension branch 140 deflects to come out of the patient's mouth, presents an external surface that covers the labial commissure of the mouth.

Moreover, at least one free end region of the extension branch 140 comprises at least one support element 142, 143 which is adapted to support at least one position reference element.

In the invention, the position reference element is designed to serve as a guide element for a navigation system for dental surgery of known type.

By “navigation system for dental surgery” it is meant a computer-assisted navigation system which makes it possible to provide visual assistance to a practitioner during a dental procedure. Several guidance technologies exist (e.g. by optical or electromagnetic fluoroscopy), but the aim remains the same, namely to provide, using the medical imaging, accurate feedback of the position of the dental stabilization block 100.

In practice, the position reference element is adapted, when the position reference element is moved in response to at least one movement of the patient's head, to trigger a registration of one or several dental images associated with the patient, based on the movement of the patient's head.

The invention also covers a first dental stabilization system.

In practice, the first dental stabilization system comprises the dental stabilization block 100 according to the second embodiment of the third way of carrying out the invention and a position reference element as described above.

Particularly, the position reference element is configured to be coupled to at least one support element 142, 143 of the free end region of the extension branch 140.

The invention a also covers second dental stabilization system.

In practice, the second dental stabilization system comprises two dental stabilization blocks 100 according to any one of the variants of the invention.

Particularly, the two dental stabilization blocks 100 of the second dental stabilization system are configured to be positioned on opposite sides of the patient's mouth.

Finally, as illustrated in [FIG. 10], the invention covers a method 200 for dental stabilization during a dental procedure.

First of all, the method 200 comprises a step of installing 210, in the patient's mouth, at least one dental stabilization block 100 according to any one of the variants of the invention, on upper and lower molars and/or premolars of a patient, hereinafter called “teeth”.

Then, the method 200 comprises a step of introducing 220 an end portion of a device for injecting a bite registration material to the inlet orifice 1131, the bite registration material being intended to harden and to be molded at least on the teeth.

Finally, the method 200 comprises a step 230 of injecting the bite registration material into the inlet orifice 1131.

The invention has been described and illustrated. However, the invention is not limited to the embodiments that have been presented. Indeed, numerous combinations of the variants, alternatives, embodiments and implementations can be envisaged without requiring substantial modifications to the invention. Thus, an expert in the field can deduce other variants, alternatives, embodiments and implementations upon reading the description and the appended figures and depending on the economic, ergonomic and dimensional constraints to be met.

The invention may be the subject of numerous variants and applications other than those described above. Particularly, unless otherwise indicated, the different structural and functional characteristics of each particular implementation described above should not be considered as combined and/or closely and/or inextricably related to each other but, on the contrary, as simple juxtapositions. Furthermore, the structural and/or functional characteristics of the different embodiments described above may be the subject in whole or in part of any different juxtaposition or any different combination.

1. A dental stabilization block intended to be bitten, only on one side of a mouth, between upper and lower molars and/or premolars of a patient, called teeth, to keep the patient's mouth open during a dental procedure so as to prevent the teeth from meeting, thereby authorizing access to teeth and/or gums on an opposite side of the mouth and at a front of the mouth, without being substantially hampered by the dental stabilization block, the dental stabilization block comprising:
a body, of essentially parallelepiped shape, which comprises, when the dental stabilization block is in place in the patient's mouth,
an upper wall, having an upper outer tightening surface which is designed to come into contact with the upper teeth, the upper outer tightening surface comprising at least a first outlet orifice,
a lower wall, having a lower outer tightening surface which is designed to come into contact with the lower teeth, the lower outer tightening surface comprising at least a second outlet orifice,
a first side wall which is oriented towards an inside of the mouth,
a second side wall which is oriented towards an inner face of a cheek,
a front end wall which is oriented towards the front of the mouth,
a rear end wall which is oriented towards a back of the mouth,
at least a first inner channel which opens out towards outside of the body at a level of at least the first outlet orifice and/or at least the second outlet orifice, the first inner channel defining a flow path which is configured to be traversed by a bite registration material intended to harden and to be molded at least on the teeth,
at least a second inner channel which opens out towards the outside of the body at a level of an inlet orifice which is configured to receive at least one end portion of a device for injecting the bite registration material, the inlet orifice being disposed on an outer surface of the first side wall and/or the front end wall, and
a first inner junction region which is configured to establish fluid communication between the first inner channel and the second inner channel.

2. The dental stabilization block according to claim 1, further comprising,
said at least a first inner channel comprising a plurality of first inner channels arranged so that, in response to the injection of the bite registration material, the bite registration material arrives substantially at the same time at the level of the outlet orifices, and
a plurality of second inner junction regions arranged so that each second inner junction region establishes fluid communication between at least two of the first inner channels.

3. The dental stabilization block according to claim 1, wherein the bite registration material is chosen from: waxes, zinc oxide-eugenol pastes, addition silicone elastomers, polyester elastomers and any combinations thereof.

4. The dental stabilization block according to claim 1, wherein the first side wall and the second side wall are designed to each extend towards a top and/or towards a bottom of the mouth beyond the upper outer tightening surface and/or the lower outer tightening surface to cover the upper and/or lower teeth so as to form an upper enclosure and/or a lower enclosure of U-shaped section in which the bite registration material and upper and/or lower teeth can be retained, a base of the U-shaped section of the upper enclosure or of the lower enclosure containing, respectively, the upper outer tightening surface or the lower outer tightening surface, wings of the U-shaped section of the upper enclosure or of the lower enclosure containing, respectively, the first side wall and the second side wall.

5. The dental stabilization block according to claim 1, wherein the second side wall is provided with a cheek retractor part which is intended to retract an inner cheek face of the second side wall, the cheek retractor portion protruding from the second side wall towards the inner cheek face and, when the dental stabilization block is in place in the patient's mouth, has an L shape with a rounded bend, one of whose branches, called bearing branch, and the rounded bend bear on the inner cheek face and whose other branch, called cantilever branch, is cantilevered and bears, at least partly, on the rear end wall.

6. The dental stabilization block according to claim 5, wherein the cheek retractor part comprises at least a third inner channel which is arranged, at least partly, inside the rounded bend, the third inner channel opening out towards the outside of the body of the bearing branch at a level of a suction orifice and at a level of a discharge orifice, the suction orifice being disposed on the cantilever branch, the discharge orifice being disposed on the bearing branch.

7. The dental stabilization block according to claim 6, wherein the bearing branch comprises a suction cannula guide part which is open in a direction of the second side wall and which extends axially from the discharge orifice to a free end of the bearing branch.

8. The dental stabilization block according to claim 1, wherein the upper outer tightening surface and the lower outer tightening surface are provided with a plurality of fluid flow disturbance ribs.

9. The dental stabilization block according to claim 5, further comprising an extension branch which extends obliquely or perpendicularly from a free end of the bearing branch towards an outside of the mouth, the extension branch being designed to form a direction change region which, when the extension branch deflects to come out of the patient's mouth, has an external surface that covers a labial commissure of the mouth,
wherein, at least one free end region of the extension branch comprises at least one support element which is adapted to support at least one position reference element which is designed to serve as a guide element for a navigation system for dental surgery, the position reference element being adapted, when the position reference element is moved in response to at least one movement of the patient's head, to trigger a registration of one or several dental images associated with the patient, based on the movement of the patient's head.

10. The dental stabilization system comprising:
the dental stabilization block according to claim 9, and
a position reference element which is configured to be coupled to at least one support element of the at least one free end region of the extension branch, the position reference element being designed to serve as a guide element for a navigation system for dental surgery, the position reference element being adapted, when the position reference element is moved in response to at least one movement of the patient's head, to trigger a registration of one or several dental images associated with the patient, based on the movement of the patient's head.

11. A dental stabilization system comprising:
two dental stabilization blocks according to claim 1, the dental stabilization blocks being configured to be positioned on opposite sides of the patient's mouth.

12. A method for dental stabilization during a dental procedure, the method comprising:
a step of installing, in the patient's mouth, at least one dental stabilization block according to claim 1, on upper and lower molars and/or premolars of a patient, called teeth,
a step of introducing an end portion of a device for injecting a bite registration material to the inlet orifice, the bite registration material being intended to harden and to be molded at least on the teeth, and
a step of injecting the bite registration material into the inlet orifice.
